(19) United States
(12) Patent Application Publication
(10) Pub. No.: US 2023/0081986 A1
(43) Pub. Date: Mar. 16, 2023

(54) METHOD AND REAGENTS TO IMPROVE NONANIMAL OCULAR TOXICITY TESTS

(71) Applicants: Stewart Lebrun, Anaheim, CA (US); Linda Nguyen, Orange, CA (US)

(72) Inventors: Stewart Lebrun, Anaheim, CA (US); Linda Nguyen, Orange, CA (US)

(73) Assignee: Lebrun Labs LLC, Anaheim, CA (US)

(21) Appl. No.: 17/889,026

(22) Filed: Aug. 16, 2022

Related U.S. Application Data
(60) Provisional application No. 63/240,235, filed on Sep. 2, 2021.

(51) Int. Cl. G01N 33/50

(52) U.S. Cl. CPC G01N 33/5058; G01N 33/5014; G01N 33/5082

(57) ABSTRACT

Disclosed are formulations and procedures to improve the accuracy of nonanimal tests. Disclosed procedures involve both the direct application of the substance to be tested to the excised eye or other suitable test matrix, such as a differentiated tissue, and the application of an aqueous layer to the apical surface and then the addition of the substance to be tested as an overlay to the aqueous layer for a period of time so as to allow metabolism of the substance to be tested by the eye or test matrix, but not so long as to result in nonirritant or nontoxic test substance resulting in a FP result.

### BACKGROUND

Testing and classification of chemicals or mixtures that cause adverse ocular effects are routinely conducted to ensure that materials are appropriately classified, labeled, and meet regulatory and safety guidelines [1]. The current benchmark ocular irritation test is the in vivo Draize rabbit eye test that uses live rabbits and a clinical scoring system to grade the severity of irritation over time based on external ocular effects to the cornea, iris, and conjunctiva until effects cannot be observed. Based on these responses, chemicals are classified using either European Union Globally Harmonized System (GHS) or U.S. Environmental Protection Agency (EPA) classification criteria. The GHS classes include nonclassified (NC, no serious eye damage, averaged over days 1, 2, and 3 ), 2 (2 A, irritation effects are reversed within 21 days; 2 B, within 7 days), and 1 (extreme and/or irreversible irritation effects that are not reversed within 21 days). The EPA classes include IV (no significant damage after 24 hours of exposure), III (damage is reversed within 7 days of exposure), II (damage is reversed within 21 days of exposure), and I (corrosive, lesions are not reversed within 21 days) [2].

Currently, there is widespread public opinion against the use of live animals for routine product testing, and there are limitations on the use of animals for product testing in many countries [3, 4]. At present, legislation in the U.S. proposes to ban the use of animals for a wide range of testing applications [5], while at the same time requiring EPA to evaluate all existing and new chemicals for unreasonable risk of injury to human health or the environment [6]. In addition, the California Cruelty-Free Cosmetics Act (SB 1249), which passed Sep. 28, 2018, made California the first state in the U.S. to ban the sale of cosmetics if animal testing is used to determine the safety of the product or its formulation by the manufacturer or its suppliers [7].

At present, there are a limited number of nonanimal ocular eye irritation tests (EITs) that have been recognized by the Organization for Economic Cooperation and Development (OECD) for which test guidelines have been established.

1) Cell culture-based tests in which test substances are applied to cells and cytotoxicity [Short Time Exposure (STE) test] or loss of barrier function measured by fluorescein permeability [Fluorescein Leakage (FL) test] is determined. While the STE test can detect 84.6% of GHS NC and 83.2% of GHS 1 materials, the FL test cannot detect NC chemicals and only detects 77.5% of GHS 1 materials. Furthermore, both tests have high false-negative (FN) rates as high as 51.3%, and neither can detect REVERSIBLE IRRITATION.

2) Tissue-based tests in which food-source animal eyes [Isolated Chicken Eye (ICE) and the Bovine Corneal Opacity and Permeability (BCOP) tests] are used. For both tests, materials are applied to the corneal surface, and damage is assessed by measuring corneal opacity, swelling (ICE), and fluorescein staining (ICE) or fluorescein permeability (BCOP). Again, both tests show 87.5% and 68.9% accuracies for detecting GHS NC materials and 82.6% and 78.5% accuracies for detecting GHS 1 chemicals, respectively, but also show high false-positive (FP) and FN rates and cannot detect REVERSIBLE IRRITATION.

3) Reconstituted human corneal epithelial (rhCE) tests in which materials are applied to three-dimensional epithelial cultures of human skin, cornea, and limbal corneal epithelial cells (EpiOcular™, SkinEthic™, and MCTT HCE™ EIT, respectively), and then cell survival is measured. These tests exhibit 80.0%, 84.0%, and 86.0% accuracies for detecting GHS NC chemicals, respectively, have very high FP rates, fail to detect GHS 1 materials, and cannot detect REVERSIBLE IRRITATION.

Attempts to address limitations in irritant or toxicity detection have generally focused on combining tests (tiered testing) [8, 9] and using “defined approaches” [10]. However, using known results to improve outcomes is primarily a post hoc statistical strategy that statistically reduces sensitivity [11]. Furthermore, we have recently shown that current EITs misclassify many of the same materials, dramatically limiting any improvement in irritant or toxicity detection that could result from tiered-testing strategies, essentially making them no different than using a single test [11]. Taken together, the limitations of current alternative tests suggest that nonanimal tests have missing variables, making them imperfect models of the live eye. Missing variables explains the high FP rate of nonanimal tests to detect nonirritants or toxicity, the high FN rate of nonanimal tests to detect ocular corrosives, and the inability of nonanimal tests to detect reversible irritants or toxicity—those that cause damage and irritation to the eye, but then clear the 21st day after the exposure.

### SUMMARY

Disclosed are formulations and procedures to improve the accuracy of nonanimal tests. Disclosed procedures involve both the direct application of the substance to be tested to the excised eye or other suitable test matrix, such as a differentiated tissue, and the application of an aqueous layer to the apical surface and then the addition of the substance to be tested as an overlay to the aqueous layer for a period of time so as to allow metabolism of the substance to be tested by the eye or test matrix, but not so long as to result in nonirritant or nontoxic test substance resulting in a FP result. In one embodiment, the aqueous layer contains salt, proteins, and antioxidants to model physiological tears. In another embodiment, toxicity after metabolism is confirmed by the addition of a metabolic inhibitor, for example, an esterase inhibitor or inhibitor of another enzyme likely to be involved in the conversion of nontoxic materials into toxic materials, such as an inhibitor of cytochrome P450. By testing metabolism, an important mechanism of toxicity is accounted for: Despite over 20 years of nonanimal eye test development, such a mechanism has never been recognized as part of a nonanimal eye irritation test, and by its incorporation, the FN rate is decreased, resulting in a greatly improved eye irritation toxicity strategy and results, as measured by accuracy and sensitivity.

### BRIEF DESCRIPTION OF THE DRAWINGS

FIG. 1 depicts the esterase catalysis of Methyl cyanoacetate into Cyanoacetic acid.

FIG. 2 depicts the effects of ebelactone on rabbit cornea. FIG. 2A shows the rabbit cornea after a 6-minute exposure to water without ebelactone. FIG. 2B shows the rabbit cornea after a 6-minute exposure to water with ebelactone. FIG. 2C shows the rabbit cornea after a 6-minute exposure to methyl cyanoacetate without ebelactone. FIG. 2D shows the rabbit cornea after a 6-minute exposure to methyl cyanoacetate with ebelactone.

FIG. 3 depicts the EPA and GHS prediction models. FIG. 3A shows the GHS prediction model. FIG. 3B shows the EPA prediction model. Meta. = Metabolic; GHS = Globally Harmonized System of Classification and Labeling of chemicals; EPA = Environmental Protection Agency; NC = Not Classified; * = Apical death of epithelium of at least 10%; must be a clear line of nuclei.

FIG. 4 depicts the comparison between the Direct and Metabolic assay for two chemicals; Isopropyl acetoacetate and 2,6-Dichlorobenzoyl chloride. FIG. 4A shows the Direct assay result for Isopropyl acetoacetate. FIG. 4B shows the Metabolic result for Isopropyl acetoacetate. FIG. 4C shows the Direct assay result for 2,6-Dichlorobenzoyl chloride. FIG. 4D shows the Metabolic assay result for 2,6-Dichlorobenzoyl chloride.

### DETAILED DESCRIPTION

In Vitro Modeling of Ocular Irritation: As a routine testing lab, it has been our experience that the identification of reversible irritants or toxicity is a challenge primarily because of the FP rates of these tests, suggesting that there is a need for a more biologically relevant and mechanistic EIT that can identify reversible irritants or toxicity.

We have developed a test model as described in our publication [12] and patent application “Formulations and Methods Related to Eye Irritation, Pat. Application Number 17/203467” [13]. This method uses food-source rabbit eyes that were initially screened for damage using Lissamine green [14] and then exposed to potential ocular irritants or toxicity using a dosing ring to assess acute responses. Following the subsequent rinsing of eyes and 24 hours of organ culture, the depth of injury (DoI) to the cornea was measured histopathologically following corneal fixation, processing, and staining for biomarkers of live/dead cells.

In our recent publication [11], we identified a group of GHS NC chemicals that are generally overclassified by most, if not all, in vitro alternative ocular irritation tests as well as chemicals that are classified as FN by other nonanimal tests, including BCOP — Laser Light-Based Opacitometer (BCOP-LLBO), BCOP — Opacitometer Kit (BCOP OP-KIT), STE, EpiOcular (EPI), ICE, Ocular Irritection (OI), and OptiSafe™ (OS). Note that the FP rates for these chemicals range from 80 to 100%. We next evaluated the chemical properties of common FP chemicals by searching publication databases; we identified that many FPs exhibited chemistries that covalently bind molecules via electron transfer and redox cycling that can lead to the generation of reactive oxygen species (ROS) [15, 16] or act as a chemical crosslinker (CL). We further noted that the eye contains high levels of antioxidants in the tear film, the first barrier to chemicals interacting with the eye. In a recent study, we found that the tear antioxidant, ascorbic acid, significantly reduced the FP results when using OS [13, 17]. In our study, five tear-related antioxidants were individually added to the OS formulation, and the effects on the optical density (OD), which is used for irritant classification, were determined. Ascorbic acid, the most abundant water-soluble antioxidant found in tears [18], was the most effective tear antioxidant, reducing both the OD and, consequently, the FP classification rate compared to the other tear antioxidants tested. Titration curves showed that this reduction occurred at physiologic tear concentrations for ascorbic acid and appeared specific for chemicals identified as producing ROS or acting as a CL. We have thus modified the IVD EIT protocol to include a “tear” solution (buffered ascorbic acid, proteins, saline). The inclusion of an antioxidant “tear” wash step adds an additional proprietary element to the test system, which reduces the FP rate.

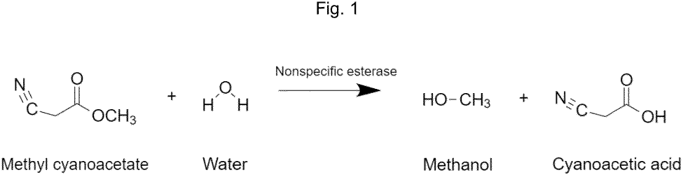

In a literature review related to our 2020 paper, we also noticed that methyl cyanoacetate was misclassified as FN for most nonanimal tests [11]. Therefore, we performed another preliminary evaluation before initiating a validation study of the method. During this initial development phase, we found that when using the procedures as described—“direct application for 1 minute” [19]—the IVD EIT classified methyl cyanoacetate as a FN. We hypothesized that the reason methyl cyanoacetate was misclassified by our IVD EIT was that the exposure time of 1 minute was too short to allow for metabolism by nonspecific esterases required for the catalysis of methyl cyanoacetate into cyanoacetic acid (FIG. 1). As shown in FIG. 1, methyl cyanoacetate contains methanol bound by an ester to cyanoacetic acid, a known ocular irritant. Since the cornea has significant nonspecific esterase activity [20], we hypothesize that additional time is required for esterases present in the eye [21, 22] to generate cyanoacetic acid and that this activity is species specific [23].

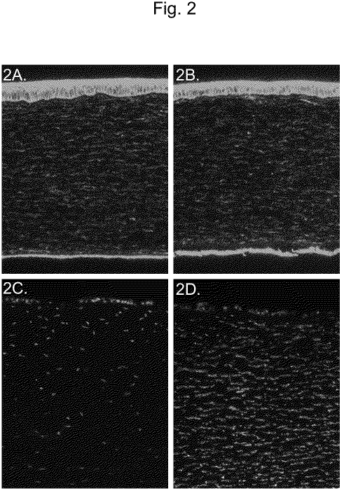

To test this hypothesis, we incubated corneas with 100 µL of the test chemical for different exposure times and observed that methyl cyanoacetate produced a toxic effect on the eye after 6 minutes of exposure. To confirm that this increased toxicity was metabolically related, eyes were treated with ebelactone, an inhibitor of nonspecific esterases [24]. FIG. 2 shows the effects of methyl cyanoacetate on the cornea after a 6-minute exposure with and without ebelactone. As shown in FIG. 2, without ebelactone, the cornea is swollen with no viable cells; however, in the presence of ebelactone, the cornea is somewhat swollen but there are mostly viable cells (green/ no DoI). Our literature review also revealed that there are a significant number of studies on the ocular metabolism, activation, and inactivation of drugs [25, 26], but we could not identify any studies on the metabolism of eye toxins or the impact on the available nonanimal tests. We thus hypothesize that species-specific metabolic activation of chemicals like methyl cyanoacetate could explain why nonanimal tests for ocular irritation have a significant FN rate and do not accurately detect reversible irritants or toxicity. Based on these findings, we updated the IVD EIT method to include the following: Each chemical is first tested using a direct 1-minute application to the eye; if negative, the chemical is then retested for 6 minutes using the metabolic test (META). All of the steps of the META test are the same as the direct test except for the dosing step. For the META, 100 µL of tear solution (saline with ascorbic acid, protein) is first added to the dosing ring, followed by the addition of 100 µL of the material to be tested. The tear solution is required to reduce the FP rate while still allowing enough time for enzyme kinetics (theoretically to convert reactants to products, as demonstrated in FIGS. 1 and 2).

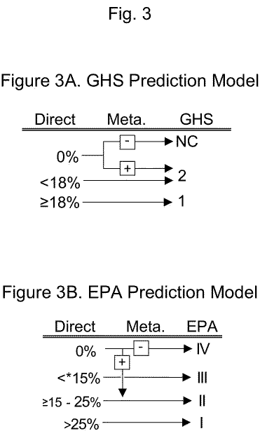

Because of these modifications, we have updated our prediction model to include the metabolic test to identify reversible irritants or toxicity as shown in FIG. 3. For GHS (A) and EPA (B) classification systems, if the direct procedure (100 µL, washed after 1 minute exposure with 20 mL tear solution using a blunt end syringe 2-5 cm from cornea to create a laminar flow across the tissue) is negative, the metabolic result is used to confirm the negative result or identify reversible irritants or toxicity (GHS 2 or EPA II) if increased DoI is detected. For the EPA system only, category III includes damage to the epithelium of the stroma up to 15%, category II includes damage greater than 15% but less than 25% (or is positive for cell death by the metabolic test), and category I includes damage greater than 25%. For the GHS classification system, the NC is defined by no stromal DoI and a negative metabolic test. Category 2 is identified by any stromal DoI up to 18% or a positive metabolic test. GHS category 1 is defined as stromal damage of 18% or greater.

With these important modifications, we next performed our prevalidation test to assess the ability of the IVD EIT to categorize 32 test substances, according to GHS and EPA classifications, and further refine the prediction model. The results for the study of 32 chemicals are shown in Table 1. The number of chemicals was selected to have a more balanced representation of each level of irritation: nonirritant (n=16), reversible irritant (n=8), and ocular corrosive (n=8).

### Validation Results

With these important modifications, we next performed our prevalidation test to assess the ability of the IVD EIT to categorize 32 test substances, according to GHS and EPA classifications, and further refine the prediction model. The results for the study of 32 chemicals are shown in Table 1. The number of chemicals was selected to have a more balanced representation of each level of irritation: nonirritant (n=16), reversible irritant (n=8), and ocular corrosive (n=8).

TABLE 1

IVD EIT Results

| Chemical Name | CASRN | in vivo EPA¹ | in vivo GHS¹ | Code No. | Direct | Meta. | Prediction | |
|---|---|---|---|---|---|---|---|---|
| | | | | | | | EPA | GHS |
| iso-Octyl acrylate | 29590-42-9 | IV | NC | 1 | 0.0% | Neg. | IV | NC |
| 1,6-Dibromohexane | 629-03-8 | IV | NC | 7 | 0.0% | Neg. | IV | NC |
| 1,2,6-Hexanetriol | 101-86-0 | IV | NC | 8 | 0.0% | Neg. | IV | NC |
| sec-Butylbenzene | 135-98-8 | IV | NC | 9 | 0.0% | Neg. | IV | NC |
| Hexane | 110-54-3 | IV | NC | 14 | 0.0% | Neg. | IV | NC |
| Dioctyl ether | 629-82-3 | IV | NC | 17 | 0.0% | Neg. | IV | NC |
| 2,4-Pentanediol | 625-69-4 | IV | NC | 18 | 0.0% | Neg. | IV | NC |
| Hexamethyldisiloxane | 107-46-0 | IV | NC | 19 | 0.0% | Neg. | IV | NC |
| Methyl amyl ketone | 110-43-0 | III | NC | 22 | 6.0% | N/A | III | 2 |
| iso-Octylthioglycolate | 25103-09-7 | IV | NC | 26 | 0.0% | Neg. | IV | NC |
| n-Octyl bromide | 111-83-1 | IV | NC | 31 | 0.0% | Neg. | IV | NC |
| Toluene | 108-88-3 | III | NC | 33 | 50.1% | N/A | I | 1 |
| Propylene glycol | 57-55-6 | IV | NC | 34 | 0.0% | Neg. | IV | NC |
| Di-iso-butyl ketone | 108-83-8 | IV | NC | 39 | 0.0% | Neg. | IV | NC |
| 3-Methoxy-1,2-propanediol | 623-39-2 | IV | NC | 40 | 0.0% | Neg. | IV | NC |
| Glycerol | 56-81-5 | IV | NC | 42 | 0.0% | Neg. | IV | NC |
| 3-Chloropropionitrile | 542-76-7 | III | 2B | 13 | 12.0% | N/A | III | 2 |
| Isopropyl acetoacetate | 542-08-5 | III | 2B | 16 | 0.0% | Pos. | II | 2 |
| Cyclopentanol | 96-41-3 | II | 2B | 30 | 30.4% | N/A | I | 1 |
| 2-Methyl-1-pentanol | 105-30-6 | III | 2B | 37 | 5.7% | N/A | III | 2 |
| 2,6-Dichlorobenzoyl chloride | 4659-45-4 | II | 2A | 15 | 0.0% | Pos. | II | 2 |
| Methyl acetate | 79-20-9 | II | 2A | 20 | 24.0% | N/A | II | 1 |
| Acetone | 67-64-1 | II | 2A | 21 | 0.0% | Pos. | II | 2 |
| Propasol solvent P | 1569-01-3 | II | 2A | 36 | 18.3% | N/A | II | 1 |
| Hydroxyethyl acrylate | 818-61-1 | I | 1 | 3 | 100.0% | N/A | I | 1 |
| Benzenesulphonylchloride | 98-09-9 | *I² | 1 | 4 | 52.8% | N/A | I | 1 |
| Diethylaminopropionitrile | 5351-04-2 | II | 1 | 5 | 56.8% | N/A | II | 1 |
| Acetic Acid (10%) | 64-19-7 | I | 1 | 6 | 100.0% | N/A | I | 1 |
| Cyclohexanol | 108-93-0 | I | 1 | 12 | 29.6% | N/A | I | 1 |
| 2,2-Dimethyl butanoic acid | 595-37-9 | I | 1 | 27 | 27.4% | N/A | I | 1 |
| Lactic acid | 50-21-5 | I | 1 | 28 | 100.0% | N/A | I | 1 |
| 2-Hydroxy isobutyric acid ethyl ester | 80-55-7 | *I² | 1 | 45 | 42.3% | N/A | I | 1 |

Table 1 includes the chemical name, in vivo classifications, direct and metabolic test results, and test prediction based on the GHS and EPA prediction models. The IVD EIT achieved 100% sensitivity for detecting all three GHS classifications with accuracies of 93.8%, 84.4%, and 87.5% for detecting NC, reversible (2), and irreversible (1) classifications of irritants, respectively. CASRN = Chemical Abstracts Service Registry Number; EPA = Environmental Protection Agency; GHS = Globally Harmonized System of Classification and Labeling of Chemicals; Meta. = Metabolic; Neg. = Negative; Pos. = Positive; N/A = Not applicable; the Metabolic assay was on run on chemicals that resulted in a 0% Direct result. * = Classification from NICEATM was “Study Criteria Not Met” (SCNM); the study was terminated on the rabbit due to extreme pain so therefore, can conclude that the EPA classification can be Category I. 1 = Lebrun et al., 2020; 2 = NICEATM, 2006

To our knowledge, this in vitro test shows the highest sensitivity for detecting all three classifications and is the first to accurately classify reversible materials (GHS Category 2 B/A and EPA Category III and II). Therefore, this test is unique and could be an invaluable tool for the accurate classification of chemicals and consumer products. The results presented exceed the accuracy of other nonanimal tests for nonirritants and ocular corrosives and is the only test with a reasonable accuracy for reversible irritants or toxicity. The test has 100% sensitivity (zero FNs) for all classes of irritation, including reversible. This is directly attributable to the simplicity of the metabolic test as disclosed. Since no other test method has incorporated a metabolic test, and all other test methods have FNs, this finding is unexpected. Metabolism of the substance to be tested is an unaccounted-for variable in all other nonanimal tests prior to the currently disclosed invention.

The specific invention overcomes deficiencies in other nonanimal eye irritation tests, allowing for the more accurate detection of reversible irritants with reduced FP and FN results. These innovations include:
1. The IVD EIT GHS prediction is based on damage to the stroma, not the corneal epithelium. We have proposed that damage to the stroma is a qualitatively different type of damage that results in a more severe irritation response because the basement membrane is damaged, thus making it harder for the epithelium to recover. Additionally, the fibroblasts of the stroma do not readily replicate, and injury may induce them to differentiate into myofibroblasts [27, 28], resulting in a more serious response. The IVD EIT method, which uses only damage to the stroma to predict GHS irritation, achieves 100% sensitivity for the GHS classification, providing strong evidence that for all of the irritants evaluated, stromal damage correlates with irritant classification. It is interesting to note that for a number of the nonirritants, there was epithelial damage, providing one explanation as to why cell-based tests tend to be overpredictive.
2. We have identified that tears contain antioxidants and other constituents that modify the chemistry and thus toxicity of chemicals contacting the eye. We found that ascorbic acid, at physiological concentrations, specifically reduces the FP rate of the OS test [13, 17]. Ascorbic acid is part of the proprietary medium and “tear solution” used to wash eyes after exposure. The IVD EIT has the lowest FP rate for commonly overpredicted chemicals.
3. We have demonstrated that esterase metabolism is required for the “activation” of some ocular toxins. To address metabolism/activation of chemicals, the method includes both a “direct application” condition and a “metabolic application” (longer exposure time but diluted in our proprietary ascorbic acid/proteins/saline “tear” solution). Using this procedure, the method detects irritants or toxicity that are FN by other nonanimal tests. The combination of the direct and metabolic procedures results in a zero FN rate for the test method.

Overall, we propose that the disclosure is innovative because it is a better biological and statistical match to the benchmark: 1) The same species/strain can be used; 2) the same tissue (cornea) can used; 3) the tearing process and tear antioxidants to lower the FP rate are included as part of the test procedure; 4) the test has a 24-hour postexposure period under organ culture conditions that better match the in vivo irritation timepoints; and 5) species-specific metabolism is specifically addressed to lower the FN rate. Using these procedures, this test outperforms all other nonanimal tests; its high sensitivity and accuracy are attributed to using the disclose inventions related to metabolism.

### In Vitro Depth of Injury Eye Irritation Test (IVD-EIT) (Lebrun Labs LLC Procedure)

Only food source eyes are suitable for this procedure. Ensure proper documentation that the eyes are extra eyes from a food processing facility before placing the order. The day before the eyes arrive, place one bottle of antioxidant medium and one bottle of 1x PBS antioxidant buffer solution (Ascorbic Acid; 1.70 mM (0.3 mg/ml)) into the incubator and place one bottle of 1x PBS antioxidant buffer solution (Ascorbic Acid; 1.70 mM (0.3 mg/ml)) into the refrigerator.

### Receiving the Eyes

1) Remove packing slip from the box and fill out the receiving form with the necessary information.

2) Retrieve a large Pyrex dish and place one ice pack in it.

3) Open the box and open the Styrofoam box that contain the eyes and take out the bag containing the eyes in jars. Remove the jars and immediately place on the ice packs in the large Pyrex dish prepared.

### Eyelid Removal

1) Place a large piece of gauze on the plastic-lined pad in the work area.

2) Remove one eye from the jar and place on the gauze.

3) Use the scissors to make an incision behind the eyelids where it meets the sclera and cut until the whole eyelid is removed from the eyeball.

4) Use the scissors to cut the connective tissue, following the curved shape of the cornea but without touching it. Note: Do not remove too much connective tissue or it will be difficult to hold the eye when rinsing after dosing.

5) Place the eye in the “good” eye jar with refrigerated 1x PBS (Jar #1).

6) Continue until all eyes have the eyelids and connective tissue removed.

7) Place an eye holder in a small Pyrex dish.

8) Using a plastic pipette, fill the eye holder with Lissamine green dye.

9) Take the 18-gauge needle and syringe and remove the needle to aspirate the refrigerated 1x PBS.

10) Take an eye and dip the cornea into the Lissamine green dye in the eye holder and slowly move the eye around to ensure the cornea is completely covered.

11) Using the 18-gauge needle and syringe filled with the refrigerated 1x PBS, wash the Lissamine green dye off over the waste container.

12) Inspect the corneas for any Lissamine green dye indicating damage.

13) Place the undamaged eyes in a new jar (Jar #2) filled with fresh refrigerated 1x PBS and damaged eyes in a separate container to discard.

### Preincubation

1) Open the 12 well plate(s) and fill each well with a “good” eye ensuring the cornea is facing up and the optic nerve is at the bottom of the well.

2) Add varying amounts of warm media from the incubator to each well ensuring the limbus is not covered. Note: The amount will vary per well due to inconsistent sizes of the eyes.

3) Put the lid onto the 12 well plate(s) and place into the incubator (37° C., 5% CO2) for one hour.

### Dosing

1) After incubation, remove the 12 well plate(s) from the incubator and place into the hood.

2) Label the wells on the lid with what is being tested.

3) Place an eye on the holder and carefully place a new cloning ring in the middle of the cornea without scratching it.

4) Fill the syringe with 40 mL of refrigerated 1x PBS antioxidant buffer solution (Ascorbic Acid; 1.70 mM (0.3 mg/ml)) by sucking it up from the beaker

5) Put the needle back on the syringe by twisting on, and then remove the cap that protects the needle.

6) Place the syringe flat on a sterile piece of foil.

7) Set the timer for “counting up”.

8) Add by micropipette first 100 µL of antioxidant buffer solution (Ascorbic Acid; 1.70 mM (0.3 mg/ml)) then 100 µL of test substance

9) Start the timer for one minute.

10) After the one minute is over, remove the dosing ring and put in the proper container, and pick up the tissue without touching the cornea and hold it over the container for liquid waste.

### Washing and Postincubation

1) Directly dispense 20 mL of antioxidant buffer solution (Ascorbic Acid; 1.70 mM (0.3 mg/ml)) onto the area of the cornea that was exposed to the sample as well as the back of the eyeball to ensure all the sample is washed off.

2) Place the eye back into its original well.

3) Continue for the rest of the eyes and samples.

4) Once all eyes are dosed, label a new 12 well plate and transfer the eyes into it.

5) Then add fresh media to the new 12 well plate(s).

6) Place the 12 well plate(s) into the incubator at 37° C. + 5% CO2 for 24 hours.

### Cornea Extraction and Fixation

1) Retrieve the correct number of 12 well plate(s) and label the lid above each well with the correct dosing test substance.

2) Fill each well with 5 mL of the 4% paraformaldehyde

3) Once the 24-hour incubation is complete, remove the plate(s) from the incubator and place near cutting area.

4) Remove an eye and hold using the 2x2 gauze with the cornea facing up and ensuring the gauze does not cover the cornea or limbus.

5) Using the scalpel, poke a hole in the sclera about 2 mm from the cornea.

6) Using the scissors, cut along the edge of the cornea ensuring a 2 mm border of sclera.

7) Make sure the cuts are smooth and ensure the cornea does not come into contact with the scissors or the cut mat.

8) Once the cornea is separated from the rest of the eye, place the cornea with the iris attached directly into the paraformaldehyde and slowly move back and forth to get rid of any folding.

9) Continue until all corneas have been removed.

10) Saran Wrap the plate and then put the lid on

11) Put the plate(s) in the fridge.

### Sucrose Infiltration

1) Label a new 12 well plate.

2) Remove the plate(s) with the corneas suspended in 4% paraformaldehyde from the fridge.

3) Place a cutting mat on the bench and a large gauze pad down.

4) Remove each cornea one by one and use the small forceps to remove the iris.

5) Cut the cornea in half.

6) Once the cornea is cut in half, place the cornea back into the correct well of paraformaldehyde.

7) Get a new 12 well plate and fill each well with 5 mL of 10% sucrose solution.

8) Transfer the corneas from the 4% paraformaldehyde solution into the new 12 well plate containing the 10% sucrose.

9) Let sit for 15 minutes.

10) During the 15 minutes, prepare the 2:1 10% sucrose:30% sucrose solution in a 50 mL conical tube.

11) Once the 15 minutes is done, use a serological pipette to remove the 10% sucrose from the wells.

12) Fill the same wells with 4.5 mL of the 2:1 10% sucrose:30% sucrose solution.

13) Let sit for 15 minutes.

14) During the 15 minutes, prepare the 1:1 10% sucrose:30% sucrose solution.

15) Once the 15 minutes is done, use a serological pipette to remove the 2:1 10% sucrose:30% sucrose solution from the wells.

16) Fill the same wells with 5 mL of the 1:1 10% sucrose:30% sucrose solution.

17) Let sit for 15 minutes.

18) During the 15 minutes, prepare the 1:2 10% sucrose:30% sucrose solution.

19) Once the 15 minutes is done, use a serological pipette to remove the 1:1 10% sucrose:30% sucrose solution from the wells.

20) Fill the same wells with 4.5 mL of the 1:2 10% sucrose:30% sucrose solution.

21) Let sit for 15 minutes.

22) Once the 15 minutes is done, remove the 1:2 10% sucrose:30% sucrose solution from the wells.

23) Fill the same wells with 5 mL of 30% sucrose (#1).

24) Let sit for 15 minutes.

25) Once the 15 minutes is done remove the 30% sucrose solution and replace it with another 5mL of 30% sucrose solution (#2).

26) Let sit for 15 minutes.

27) Once the 15 minutes is done remove the 30% sucrose solution and replace it with another 5 mL of 30% sucrose solution (#3).

28) Let sit for 15 minutes.

### OCT Embedding and Liquid Nitrogen Freezing

1) Label all pink bags with your name, date, sample name, and Lot #.

2) Label 1 large white bag with name, date, samples and # of each, and Lot #.

3) Take out the correct number of Tissue-Tek cryomolds, and forceps.

4) Fill the first square of the Tissue-Tek cryomold with OCT.

5) Grab the outer round edge of the cornea with the forceps and place the straight edge into the OCT so it lays along the bottom of the cryomold.

6) Ensure the cornea is completely vertical and not bent.

7) Inspect from above and the sides to ensure it is in the correct orientation.

8) Rest the cryomold on the coat hanger holder and slowly insert the cryomold into the Dewar Flask until only the bottom of the cryomold is exposed to the liquid nitrogen.

9) DO NOT LET THE LIQUID NITROGEN TOUCH THE OCT DIRECTLY.

10) Once the bottom of the cryomold is in the liquid nitrogen, you should be able to see the OCT from above go from clear to completely white.

11) Once the OCT above the liquid nitrogen is completely white and frozen and has no air bubbles, the entire cryomold can be submerged into the liquid nitrogen and kept there for 20 seconds to ensure adequate freezing.

12) After the 20 seconds is finished, use the coat hanger mold to remove the cryomold and ensure all excess liquid nitrogen is poured into the Dewar Flask.

13) The entire mold can then be placed in a Pyrex dish for a couple of seconds.

14) Immediately transfer the entire cryomold with the frozen cornea in it, to its respective labeled pink bag.

15) Immediately place the pink bag into the large white bag in the -80° C. freezer for storage.

### Cryosectioning

1) Lay out paper towel to put the slides on.

2) Label the slides using the following labeling system:

3) Once all slides are labelled, lay them out on the paper towel in an organized manner.

4) Turn the light on in the cryostat.

5) Ensure the cryostat is at -25° C.

6) Ensure the small chuck is in the cryostat and in the proper freezing area on the left.

7) Ensure the 1 g metal weight is in the cryostat and at the correct temperature (if not in the cryostat, place the weight in the cryostat and wait for it to cool down to the -25° C. temperature).

8) Put the desired paint brush in the cryostat to make sure it gets to the proper temperature.

9) Retrieve a bottle of OCT if not on top of the cryostat already.

10) Check the micron setting to make sure it is set to the correct micron value.

11) Retrieve the first sample from the -80° C. freezer.

12) Take the frozen sample (still in the Tissue-Tek cryomold) and remove it from the pink bag.

13) Place the pink bag in the cryostat and the sample on top of the pink bag.

14) Close the cryostat lid and leave the sample until it is able to be popped out of the mold with ease (approximately 5-8 minutes).

15) When the sample is ready to be popped out, flip the mold upside down so when the sample is popped out it will land directly on the pink bag. The top of the frozen sample (the part exposed to air while in the mold) should be touching the pink bag. The bottom of the frozen sample (the part touching the inside of the mold which has the flat side of the cornea) should be facing upwards so you can slightly see the outline of the sample.

16) Once the sample is popped out of the mold, leave it in the orientation.

17) Get the OCT and squeeze it onto the chuck, starting from the center and dispensing in a circular motion following the rings on the chuck until the entire chuck has a layer of OCT on it.

18) Quickly pick up the frozen sample and place it on the OCT.

19) Get the 1 g metal weight and carefully place it on top of the frozen sample so it adheres to the OCT and freezes completely horizontal and flat to allow for proper cutting.

20) Close the lid and wait for the OCT to completely freeze and bond to the frozen sample (approximately 5 minutes).

21) Once the OCT is completely frozen and the sample is ready to be cut, open the lid and remove the 1 g weight.

22) Lift the chuck up and insert it into the block and tighten to secure it.

23) Use the buttons on the left-hand side to adjust the distance of the sample from the blade.

24) Once the sample is in the correct position/distance from the blade, slowly turn the handle to see if further adjustment is needed.

25) In the beginning, only certain parts of the sample may be getting cut (like the middle, bottom, or edge) — this is normal.

26) Continue slowly turning the handle to trim the frozen sample and eventually trim to the point where the entire frozen sample is being cut evenly. As sections (full or partial) are being cut, lift the glass and use the paint brush to wipe away the excess to have a clean slate for future sections.

27) Once the sections being cut are full squares and the entire cornea is being included in a section, it is ready to be put on slides.

28) Use the paint brush to wipe away any sections and start with a clean area.

29) Turn the handle to get a clean full section.

30) Lift the glass to expose the section. If the section is slightly curled or scrunched, use the paint brush to straighten it out by lightly putting the edge of the bristles on the bottom part of the section and gently pulling towards yourself to remove the wrinkles.

31) Get the slide and flip it over so the part where the section sticks is facing down.

32) Hover the slide over the section in the exact spot it will be placed on the slide.

33) Slowly put the bottom edge of the slide (closest to you) on the bottom of the section and quickly roll the slide towards the top of the slide (closest to the blade) to ensure the section gets placed on the slide without air bubbles or folds.

34) Once the section is attached to the slide, flip it over so the side that the section is on is faced up.

35) Place the slide back on the paper towel.

36) Put the glass back down gently so you can continue cutting more sections.

37) Continue cutting sections one by one and adding them to each slide closest to the plus signs.

38) Once all slides of the sample have 1 section on it, trim the sections about 100 microns by turning the handle 15 times.

39) Use the paint brush to wipe away the sections and provide a clean area.

40) Cut sections one by one again and add them to the middle of the slide until each slide for the sample has a middle section on it.

41) Trim the section again by 100 microns and use the paint brush to wipe away the sections.

42) Cut sections one by one again and add them to the top of the slide until each slide for the sample has a top section on it. Each slide should have 3 sections on it now.

43) Once all slides have 3 sections on them the cutting is done.

44) Remove the chuck from the block and leave it outside of the cryostat (approximately 1 minute) to slightly thaw the sample so it can be removed from the chuck.

45) Once the sample has been slightly thawed, remove it from the chuck and place it back into the pink bag and then the -80° C.

46) Either stain the slides in the same day, or sore the slides in a slide box. Slide box must be properly labeled using labeling tape regardless of same day staining or not.

### Staining

1) Lay out the slides, face up, in the slide box.

2) So that the top and bottom edge (white writing part and plus sign part) are laying on the groove where the slides would be put into.

3) If the slides came from the fridge, let them get to room temperature (approximately 10 minutes).

4) Put the Phalloidin and DAPI stock solution tubes on the cooling rack and keep them covered with foil to protect them from light.

5) Prepare the phalloidin mixture (this is enough to cover 16 sections). Dispense 800 µL of 1x PBS into the microcentrifuge tube. Dispense 20 µL of phalloidin into the 1x PBS. Invert to mix and place the conical tube in the cooling rack (covered with foil) until ready to dispense the solution on the sections. Phalloidin is light sensitive so keep covered at all times.

6) Dispense 50 µL on each individual cornea section.

7) Once all sections are covered with the Phalloidin solution, close the lid of the slide box but do not completely close it - just let the lid lay closed.

8) Get the foil and cover the slide box to prevent any light from going through.

9) Turn off the lights in the room to help with light issues.

10) Let the solution sit for 20 minutes.

11) Once the 20 minutes is done, remove the foil and open the lid.

12) Take each slide one by one and dump the phalloidin liquid in the waste beaker.

13) Once the excess phalloidin has been removed, use a disposable pipette to rinse the slide off with 1x PBS. Dispense the 1x PBS at the top of the slide and let the PBS run down the slide to rinse it. DO NOT dispense the PBS directly on the sections or they could come off or get ruined.

14) Once all slides have been rinsed, lay them back in the slide box and lay the lid closed so the DAPI solution can be made.

15) Prepare the DAPI mixture (this is enough to fill 1 conical tube that holds 5 slides). Fill the 50 mL conical tube with 50 mL of 1x PBS. Dispense 10 uL of DAPI into the conical tube. Put the lid on and shake until it is all incorporated. DAPI is light sensitive so keep covered at all times.

16) Pour the DAPI solution into the Coplin jar and transfer the slides into the jar.

17) Cover the Coplin jar with foil and turn the lights off once again.

18) Keep the slides in the solution for 5 minutes.

19) Once the 5 minutes is up, take the slides out one by one and use a disposable pipette to rinse off the excess DAPI solution with 1x PBS.

20) Once the slides have been rinsed off, place them lying flat in the slide box once again.

### Cover Slip Addition

1) Place 2 stacked Whatman paper on the table and keep another single paper on the side out of the way.

2) Dispense about 3 drops of glycerol on each individual cornea section .

3) Slowly place a cover slip onto the slide starting from one end and slowly angling the cover slip even more until the entire thing is on the slide. Dropping the cover slip fast will cause the formation of air bubbles and may ruin the section due to potential movement.

4) Once the cover slip is completely on, pick up the slide and place the side on its side at about a 45-degree angle on the paper (slide is facing the paper and the cover slip side is facing towards you).

5) Slowly increase the angle of the slide by bringing the top side towards you until it makes a 90-degree angle with the Whatman paper.

6) This will make a seal between the Whatman paper and the slide and cause the excess 50% glycerol to be absorbed by the Whatman paper.

7) Once the excess 50% glycerol is removed, flip the slides over to the other side and hold it against the Whatman paper at a 90-degree angle to remove any excess 50% glycerol from that end.

8) Lay the slide on the slide box again face up and use the Whatman paper left on the side to absorb any excess 50% glycerol at the edges by carefully holding the edge of the paper to the edge of the coverslip on the slide.

9) Once all of the excess 50% glycerol has been removed, the slides are ready for imaging.

10) Keep them in the slide box with the lid closed to prevent unnecessary light exposure.

### Imaging

1) Turn on the microscope (black switch on the right side).

2) At the base of the microscope the word “set” will show up in red indicating the microscope is on.

3) Turn on the fluorescence light on the power box to the left of the microscope.

4) Give the light about 3 minutes to warm up.

5) While the light is warming up, click on the “DoI Images” folder on the desktop.

6) Create a new folder within labeled “Experiment dd-mm-yy Intact Cornea 24 hour_{_} Series”

7) Within that folder, make a folder for each sample that was tested.

8) Within the folder for each sample make two folders each labeled “100x” and “400x”

9) Open the Thor Camera software and click the down arrow on the left of the screen that pops up.

10) Click “CS235MU”

11) Once the imaging window comes up, maximize the screen.

12) Put a slide in the microscope and align the 10x lens.

13) Under the eye piece there is a silver disc embedded into the microscope that can rotate to change the fluorescence filters (4 settings labeled - 1, 2, 3, & 4). 3 — FITC — DAPI

14) Rotate the disc to filter 3 and look through the eye piece - the cornea should be illuminated green with the epi being brighter green than the stroma. This means the phalloidin stained the cornea adequately.

15) Rotate the disc to filter 4 and look through the eye piece - the cornea should be illuminated blue with the nuclei in the epi a defined blue. This means the DAPI stained the cornea adequately.

16) Now that the stain has been checked it is ready to be imaged.

17) Keep the disc that controls the filter at number.

18) Starting with DAPI makes it easier and faster to focus the image.

19) Ensure the 10x lens is being used.

20) While looking through the eye piece, position the cornea where you want capture the image making sure to include the epithelium, stroma, and endothelium. The cornea must be centered in the screen and completely straight. Horizontal or vertical depending on the orientation of the camera.

21) Once the cornea is set up in the correct position, pull out the metal bar out to the left of the eyepiece on the side of the microscope. Below the bar it will say “photo, photo/vis, vis” This will allow the camera to see the image instead of you through the eye piece.

22) At the top bar, click the icon that looks like 3 gears (settings button). Move the settings window to the very right so it does not block the screen where the image will be displayed.

23) Set the exposure time to 250 ms and then press enter.

24) In the top left corner click the round green button with the white triangle in it. “Live image”

25) Once the image is displayed on the screen, adjust it up/down left/right to make it centered on the screen.

26) If the image needs to be slightly rotated to make it horizontal, slowly rotate the stage.

27) Once the image in centered and positioned correctly, use the fine tune knob to focus the image until it is clear.

28) Once the image is clear, determine if the exposure time is high enough or if it needs to be adjusted. For DAPI, the nuclei should be visible and defined but not extremely bright.

29) Once the exposure is adjusted to the correct level, press the live image button once again so it stops showing the live image. The button should be a green circle with a white square in it.

30) Then press the camera icon to take a picture.

31) The camera icon and the icons near it will grey out for a little bit and then the color will come back.

32) Once the color comes back to the icons, the picture is done being taken and ready to save.

33) To save the image, press the floppy disk with the tree in the bottom right.

34) It will bring up the file explorer where you find the folder you made earlier and save it under the correct sample name. This will be saved under the 100x folder.

35) The picture will be labeled: “‘sample name’ ‘slide #’ ‘section letter’ 100x ‘DAPI or FITC’” Ex. “Water 1A 100x DAPI”

36) Once the DAPI image is saved under the 100x folder, the phalloidin image is ready to be taken.

37) DO NOT MOVE THE STAGE.

38) ONLY turn the disc to change the filter from number 4 to number 3 so the phalloidin stain can be seen.

39) Once the filter is on number 3, click the settings button once again.

40) Start off by changing the exposure to approximately 5000.

41) Click the live image button and see how bright the phalloidin stain is.

42) The goal is to have the stroma bright and clearly visible. If the epi will become over exposed if done correctly and that is acceptable.

43) Continue to adjust the exposure until the stroma is very bright and clear.

44) Once the exposure is adjusted and the image is focused, stop the live image display.

45) Click the camera icon and wait for the image to be taken.

46) Save the image in the same folder and label it correctly (in this case it will be the same label as the previous picture but will have “DAPI” switched out for “FITC”): “‘sample name’ ‘slide #’ ‘section letter’ 100x ‘DAPI or FITC’” Ex. “Water 1A 100x FITC”

47) Continue taking pictures for the rest of the sections on the slide until all 100x images are complete. Between each section, push the rod in so you can see the sections through the eye piece. When ready to image, pull the rod out again so the camera can capture the image.

48) Turn the lens to the 40x lens.

49) Turn the disc so the filter is on number 4 (DAPI).

50) Push the rod in so you can visualize the sections through the eye piece.

51) Position the light over the first section and find a straight clear section of the epithelium.

52) Pull the rod out.

53) Set the exposure to 150 ms and press enter.

54) Press the live image button and see if the image is at the correct exposure. The nuclei should not be too dark or too bright.

55) Once the image has the correct exposure and is focused, stop the live image.

56) Press the camera icon and wait for the image to be taken.

57) Press the save image button and save the image under the correct sample name and in the 400x folder.

58) Label it correctly: “‘sample name’ ‘slide #’ ‘section letter’ 400x ‘DAPI or FITC’” Ex. “Water 1A 400x DAPI”

59) Once the DAPI image is saved under the 400x folder, the phalloidin image is ready to be taken.

60) Do not move the stage.

61) Only turn the disc to change the filter from number 4 to number 3 so the phalloidin stain can be seen.

62) Once the filter is on number 3, click the settings button once again.

63) Start off by changing the exposure to approximately 2000.

64) Click the live image button and see how bright the phalloidin stain is.

65) The epithelium should be illuminated but not over exposed. It should be clear and defined.

66) Once the exposure is adjusted and the image is focused, stop the live image.

67) Press the camera icon and wait for the image to be taken.

68) Save the image in the same folder and label it correctly (in this case it will be the same label as the previous picture but will have “DAPI” switched out for “FITC”): “‘sample name’ ‘slide #’ ‘section letter’ 400x ‘DAPI or FITC’”. Ex. “Water 1A 400x FITC”. Continue taking pictures for the rest of the sections on the slide until all 400x images are complete. Between each section, push the rod in so you can see the sections through the eye piece. When ready to image, pull the rod out again so the camera can capture the image.

Only food source eyes are suitable for this procedure. Ensure proper documentation that the eyes are extra eyes from a food processing facility before placing the order. The day before the eyes arrive, place one bottle of antioxidant medium and one bottle of 1x PBS antioxidant buffer solution (Ascorbic Acid; 1.70 mM (0.3 mg/ml)) into the incubator and place one bottle of 1x PBS antioxidant buffer solution (Ascorbic Acid; 1.70 mM (0.3 mg/ml)) into the refrigerator.

The results shown in Table 2 indicate that the false positive rate is significantly reduced by the presence of antioxidant formulation for the DoI procedure compared with other test methods.

TABLE 2

| Chemical Name | CASRN | in vivo GHS | BCOP (LLBO) | BCOP (OP-KIT) | EPI | Ol | Dol |
|---|---|---|---|---|---|---|---|
| Sodium lauryl sulfate (3%) | 151-21-3 | NC | FP | FP | FP | FP | TN |
| Ethyl acetate | 141-78-6 | NC | | FP | FP | FP | FP |
| Cyclohexanone | 108-94-1 | NC | | FP | FP | | FP |
| 2-(2-Ethoxyethoxy) ethanol | 111-90-0 | NC | | | FP | FP | TN |
| 3-Phenoxy benzyl alcohol | 13826-35-2 | NC | | | FP | FP | FP |
| 2-Ethoxyethyl methacrylate | 2370-63-0 | NC | | | FP | FP | TN |
| 2,4-Pentanedione | 123-54-6 | NC | | FP | FP | | FP |
| 1-Nitropropane | 108-03-2 | NC | FP | FP | | | TN |
| Ethylene glycol diethyl ether | 629-14-1 | NC | | | FP | FP | FP |
| Styrene | 100-42-5 | NC | | | FP | TN | FP |
| 1,9-Decadiene | 1647-16-1 | NC | FP | | TN | TN | TN |
| p-Methyl thiobenzaldehyde | 3446-89-7 | NC | | | TN | | TN |
| 2,2-Dimethyl-3-pentanol | 3970-62-5 | NC | | | FP | FP | FP |
| 1,3-Di-iso-propylbenzene | 99-62-7 | NC | FP | | TN | FP | TN |
| Triphenyl phosphite | 101-02-0 | NC | | | FP | | TN |
| Triethylene glycol | 112-27-6 | NC | | | FP | | TN |
| | | | 0 TN | 0 TN | 3 TN | 2 TN | 9 TN |
| | | | 4 FP | 5 FP | 12 FP | 8 FP | 7 FP |
| | | FPR | 100% (4/4) | 100% (5/5) | 80% (12/15) | 80% (8/10) | 43.8% (7/16) |

Legend: GHS = Globally Harmonized System of classification and labeling of chemicals; NC = Nonclassified; BCOP (LLBO)/(OP-KIT) = Bovine Corneal Opacity & Permeability Laser Light-Based Opacitometer/Opacitometer Kit; EPI = EpiOcular; OI = Ocular Irritection; DoI = Depth of Injury; CNM = Criteria not met; FP = False positive; TN = True negative.

### Metabolic Procedure

Additional testing is required to differentiate nonirritants or toxicity.

NOTE: The steps of the main procedure are the same as described above. The only deviations are described below and apply to the dosing of the corneas and interpretation of results.
1) After the pre-incubation, remove the 12 well plate(s) from the incubator and place in the hood.
2) Using a skinny sharpie, label the wells on the lid (water or specific chemical).
3) Place an eye on the holder and carefully place a new cloning ring in the middle of the cornea without scratching it.
4) Remove the needle from the syringe by twisting it off. Fill the syringe with 40 mL of 1x PBS by aliquoting it from the beaker.
5) Put the needle back on the syringe by twisting on and remove the cap protecting the needle. Place the syringe flat on a sterile piece of foil.
6) Obtain the timer. Add 100 uL of the T8 tear solution in the center of the dosing ring. Ensure the entire surface is covered.
7) Retrieve 100 uL of the sample and aspirate in the center of the dosing ring, ensuring the entire surface is exposed. Start the timer set for 3 minutes.
8) After 3 minutes, remove the cloning ring and put in the proper container, pick up the eyeball without touching the cornea.
9) Use the wash filled syringe to directly dispense 20 mL of wash onto the area of the cornea that was exposed to the sample, as well as the back of the eyeball to ensure all the sample is washed off.
10) Place the eye back into its original well on the 12 well plate.
11) Repeat steps 1-10 for the time point of 6 minutes and 9 minutes.
12) Once dosing is complete, label a new 12 well plate and transfer the eyes into it.
13) Add fresh media OMG Medium to the new 12 well plate(s).
14) Place the 12 well plate(s) into the incubator at 37° C. + 5% CO2 for 24 hours.

Prediction 1 model for the metabolic results is as follows:
If there is no reduction in stromal phalloidin signal at 9 minutes, it is EPA IV/GHS NC.
If there is significant reduction in stromal phalloidin signal compared with control at 9 minutes, it is EPA III/GHS 2B.
If there is significant reduction in stromal phalloidin signal compared with at 6 minutes, it is EPA II/GHS 2A.
If there is significant reduction in stromal phalloidin signal compared with at 3 minutes, it is EPA I/GHS 1.

Prediction 2 model for the metabolic results is as follows:
If there is no reduction in stromal phalloidin signal at 9 minutes, it is EPA IV/GHS NC.
If there is significant reduction in stromal phalloidin signal compared with control at 9 minutes, it is EPA III/II and GHS 2.
If there is significant reduction in stromal phalloidin signal compared with at 6 minutes, it is NA.
If there is significant reduction in stromal phalloidin signal compared with at 3 minutes, it is EPA I/GHS 1.

Prediction 3 model for the metabolic results is as follows:
If there is no reduction in stromal phalloidin signal at 9 minutes, it is EPA IV/GHS NC.
If there is significant reduction in stromal phalloidin signal compared with control at 9 minutes, it is EPA III/II and GHS 2.
If there is significant reduction in stromal phalloidin signal compared with at 6 minutes, it is EPA III/II and GHS 2
If there is significant reduction in stromal phalloidin signal compared with at 3 minutes, it is EPA I/GHS 1.

Prediction 4 model for the metabolic results is as follows:
If there is no reduction in stromal phalloidin signal at 9 minutes, it is EPA IV/GHS NC.
If there is significant reduction in stromal phalloidin signal compared with control at 9 minutes, it is EPA III/II and GHS 2.
If there is significant reduction in stromal phalloidin signal compared with at 6 minutes, it is EPA I and GHS 1
If there is significant reduction in stromal phalloidin signal compared with at 3 minutes, it is EPA I and GHS 1

Prediction 5 model for the metabolic results is as follows:
If there is no reduction in stromal phalloidin signal at 9 minutes, it is EPA IV.
If there is significant reduction in stromal phalloidin signal compared with control at 9 minutes, it is EPA III and GHS NC.
If there is significant reduction in stromal phalloidin signal compared with at 6 minutes, it is EPA II and GHS 2.

Optional: To confirm metabolic can use specific enzyme inhibitors (for example ebelactone to inhibit esterases). In this case only results that can be inhibited are accepted as metabolic irritants. Results that are not inhibited are outside of the application domain.

### Metabolic Example

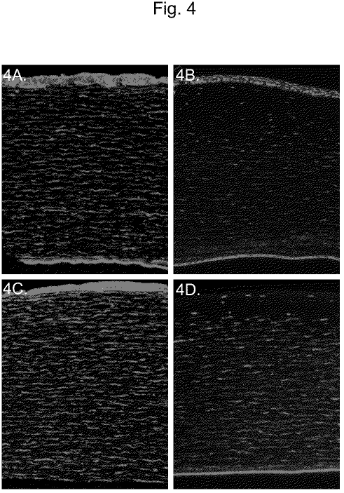

The IVD EIT test includes both a “direct assay” condition and a “metabolic assay” (longer exposure time but diluted in our proprietary ascorbic acid/protein/salt “tear” solution). Using this procedure, the method detects irritants that are FN by other nonanimal tests and we have demonstrated that esterase metabolism is required for “activation” of some ocular toxins (demonstrated for methyl cyanoacetate), likely relevant for the correct prediction of the ester Isopropyl acetoacetate (CASRN 542-08-5). In addition to esterase activity, the eye has significant cytochrome P450 enzymatic activity (Kolln and Reichl, 2016) which could have been relevant for the benzyne derivative: 2,6-Dichlorobenzoyl chloride (CASRN 4659-45-4). The combination of the direct and metabolic procedures results in a zero FN rate for the test method. The results of the direct and metabolic assay for Isopropyl acetoacetate and 2,6-Dichlorobenzoyl chloride are shown in FIG. 4.

### ACKNOWLEDGEMENTS

This invention was made with government support under (R43ES031881) awarded by NIEHS. The government has certain rights in the invention.

Research reported in this patent was supported by the National Institute of Environmental Health Sciences of the National Institutes of Health under Award Number R43ES031881. The content is solely the responsibility of the authors and does not necessarily represent the official views of the National Institutes of Health.

### BIBLIOGRAPHY

1. Bruner, L.H. 1992. Alternatives to the use of animals in household product and cosmetic testing. Journal of the American Veterinary Medical Association 200, 669-673.
2. UN. 2011. United Nations Globally Harmonized System of Classification and Labelling of Chemicals (GHS), ST/SG/AC.10/30 Rev 4, Part 3 Health Hazards - Chapter 3.3 Serious eye damage / eye irritation. United Nations Publications, New York & Geneva. http://www.unece.org/trans/danger/publi/ghs/ghs_rev04/04files_e.html.
3. Senate Joint Resolution 22. 2014. Introduced by Senator Block. http://leginfo.legislature.ca.gov/faces/billNavClient.xhtml?bill_id=201320140SJR22&search _keywords=.
4. Humane Society. 2017. Timeline: Cosmetics Testing on Animals: The Humane Society of the United States. [Online] Available at: http://www.humanesociety.org/issues/cosmetic_testing/timelines/timeline-cosmetics-testing-on-animals.html [Accessed 27 Dec. 2017].
5. H.R.2790. 2017. Humane Cosmetics Act 115th Congress. Rep. McSally, Martha [R-AZ-2] (Introduced Jun. 06, 2017) [Online] Available at: https://www.congress.gov/bill/115th-congress/house-bill/2790.
6. S.697. 2015. Frank R. Lautenberg Chemical Safety for the 21st Century Act. 114th Congress. [Online] https.7/www.congress.gov/bill/1 14th-congress/senate-bil1/697.
7. SB1249, 2018. California Cruelty-Free Cosmetics Act. Civil Code, 1934.1939.1935
8. Hayashi K., Mori T., Abo T., Koike M., Takahashi Y., Sakaguchi H., Nishiyama N. (2012). “A tiered approach combining the short time exposure (STE) test and the bovine corneal opacity and permeability (BCOP) assay for predicting eye irritation potential of chemicals”. Altern Lab Anim. 41(2): 157-171. doi: 10.1177/026119291304100205.
9. Adriaens E., Verstraelen S., Alepee N., Kandarova H., Drzewiecka A., Gruszka K., Guest R., Willoughby Sr JA., Van Rompay AR. (2018). “CON4EI: Development of testing strategies for hazard identification and labelling for serious eye damage and eye irritation of chemicals”. Toxicology in Vitro. 49: 99-115. doi: 10.1016/j.tiv.2017.09.008.
10. Alepee N., Adriaens E., Abo T., Bagley D., Desprez B., Hibatallah J., Mewes K., Pfannenbecker U., Sala A., Van Rompay AR., Verstraelen S., McNamee P. (2019). “Development of a defined approach for eye irritation or serious eye damage for liquids, neat and in dilution, based on cosmetics Europe analysis of in vitro STE and BCOP test methods”. Toxicology in Vitro. 57: 154-163. doi: 10.1016/j.tiv.2019.02.019.
11. Lebrun S., Nguyen L., Chavez S., Chan R., Le D., Nguyen M., Jester JV. (2020). “Same-chemical comparison of nonanimal eye irritation test methods: Bovine corneal permeability, EpiOcular™, isolated chicken eye, ocular Irritection®, OptiSafe™, and short time exposure”. Toxicol In Vitro. 2020: 105070. doi: 10.1016/j.tiv.2020.105070
12. Lebrun S, Xie Y, Chavez S, Chan R, Jester JV: An in vitro depth of injury prediction model for a histopathologic classification of EPA and GHS eye irritants. Toxicol In Vitro 2019 Dec; 61:104628. doi: 10.1016/j.tiv.2019.104628
13. Lebrun S., inventor. (2021). “Formulations and Methods Related to Eye Irritation”. Pat. Application Number 17/203467. In review. United States Patent and Trademark Office.
14. Hamrah P, Alipour F, Jiang S, Sohn JH, Foulks GN: Optimizing evaluation of Lissamine Green parameters for ocular surface staining. Eye 2011, 25:1429-1434.
15. van Amsterdam IMC., Ubbink M., Einsle O., Messerschmidt A., Merli A., Cavazzini D., Rossi GL., Canters GW. (2001). “Dramatic modulation of electron transfer in protein complexes by crosslinking”. Nature Structural Biology. 9: 48-52. doi: 10.1038/nsb736.
16. Kovacic P., Sacman A., Wu-Weis M. (2002). “Nephrotoxins: Widespread Roles of Oxidative Stress and Electron Transfer”. Current Medicinal Chemistry. 9: 823-847. doi: 10.2174/0929867024606803.
17. Lebrun S., Chavez S., Chan R., Nguyen L., Jester JV. (2021). “Modeling the antioxidant properties of the eye reduces the false-positive rate of a nonanimal eye irritation test (OptiSafe)”. Toxicology in Vitro. 76:105208. doi: 10.1016/j.tiv.2021.105208.
18. Chen Y., Mehta G., Vasiliou V. (2009). “Antioxidant defenses in the ocular surface”. Ocul Surf. 7(4):176-185. doi:10.1016/s1542-0124(12)70185-4
19. Lebrun S., Xie Y., Chavez S., Chan R., Jester JV. (2019). “An in vitro depth of injury prediction model for a histopathologic classification of EPA and GHS eye irritants”. Toxicol In Vitro. 61:104628. doi: 10.1016/j.tiv.2019.104628.
20. Nakamura M., Shirasawa E., Hikida M. (1993). “Characterization of Esterases Involved in the Hydrolysis of Dipivefrin Hydrochloride”. Opthalmic Res. 25:46-51. doi: 10.1159/000267220.
21. Lee VHL., Morimoto KW., Stratford Jr RE. (1982). “Esterase distribution in the rabbit cornea and its implication in ocular drug bioavailability”. Biopharmaceutics & Drug Disposition. 3(4): 291-300. doi: 10.1002/bdd.2510030402.
22.Lee VHL., Stratford Jr RE., Morimoto KW. (1983). “Age-related changes in esterase activity in rabbit eyes”. International Journal of Pharmaceutics. 13(2): 183-195. doi: 10.1016/0378-5173(83)90005-4.
23. OECD SIDS. (2006). “SIDS Initial Assessment Profile: Cyanoaceates”. SIAM. 23:17-20. Available at: https://hpvchemicals.oecd.org/ui/handler.axd?id==D8489E4D-D25A-4BED-B770-99ADA9011581
24. Hoskins IA., Katz J., Frieden FJ., Ordorica SA., Young BK. (1990). “In vitro inhibition of esterase activity in amniotic fluid: Comparison with bacterial cultures”. American Journal of Obstetrics and Gynecology. 163(6): 1944-1947. doi: 10.1016/0002-9378(90)90778-6.
25. Argikar UA., Dumouchel JL., Kramlinger VM., Cirello AL., Gunduz M., Dunne CE., Sohal B. (2017). “Do We Need to Study Metabolism and Distribution in the Eye: Why, When, and Are We There Yet?”. Journal of Pharmaceutical Sciences. 106(9): 2276-2281. doi: 10.1016/j.xphs.2017.03.008.
26. Heikkinen EM., del Amo EM., Ranta V., Urtti A., Vellonen K., Ruponen M. (2018). “Esterase activity in porcine and albino rabbit ocular tissues”. European Journal of Pharmaceutical Sciences. 123: 106-110. doi: 10.1016/e.ejps.2018.07.034.
27. Masur SK., Dewal HS., Dinh TT., Erenburg I., Petridou S. (1996). “Myofibroblasts differentiate from fibroblasts when plated at low density”. PNAS. 93(9):4219-4223. doi: 10.1073/pnas.93.9.4219.
28. Micera A., Puxeddu I., Balzamino BO., Bonini S., Levi-Schaffer F. (2012). “Chronic Nerve Growth Factor Exposure Increases Apoptosis in a Model of In Vitro Induced Conjunctival Myofibroblasts”. PLoS ONE. 7(10):e47316. doi: 10.1371/journal.pone.0047316.
29. NICEATM. (2006). “Recommended Substances for Optimization or Validation of In Vitro Ocular Toxicity Test Methods, List of Recommended Reference Substances”. Available at: https_{:}//ntp.niehs.nih.gov/whatwestudy/niceatm/resources-for-test-method-developers/refchem/index.html?utm_source=direct&utm_medium=prod&utm_campaign===ntp golinks&utm_term=refchem
30. OECD (2017), Test No. 460: Fluorescein Leakage Test Method ƒor Identifying Ocular Corrosives and Severe Irritants, OECD Guidelines for the Testing of Chemicals, Section 4, OECD Publishing, Paris, https://doi.org/10.1787/9789264185401-en.
31. OECD (2020), Test No. 491: Short Time Exposure In Vitro Test Method for Identifying i) Chemicals Inducing Serious Eye Damage and ii) Chemicals Not Requiring Classification ƒor Eye Irritation or Serious Eye Damage, OECD Guidelines for the Testing of Chemicals, Section 4, OECD Publishing, Paris, https://doi.org/10.1 787/9789264242432-en.
32. OECD (2018), Test No. 438: Isolated Chicken Eye Test Method ƒor Identifying i) Chemicals Inducing Serious Eye Damage and ii) Chemicals Not Requiring Classification ƒor Eye Irritation or Serious Eye Damage, OECD Guidelines for the Testing of Chemicals, Section 4, OECD Publishing, Paris, https://doi.org/10.1787/9789264203860-en.
33. OECD (2020), Test No. 437: Bovine Corneal Opacity and Permeability Test Method ƒor Identifying i) Chemicals Inducing Serious Eye Damage and ii) Chemicals Not Requiring Classification ƒor Eye Irritation or Serious Eye Damage, OECD Guidelines for the Testing of Chemicals, Section 4, OECD Publishing, Paris, https://doi.org/10.1787/9789264203846-en.
34. OECD (2019), Test No. 492: Reconstructed human Cornea-like Epithelium (RhCE) test method ƒor identifying chemicals not requiring classification and labelling ƒor eye irritation or serious eye damage, OECD Guidelines for the Testing of Chemicals, Section 4, OECD Publishing, Paris, https://doi.org/10.1 787/9789264242548-en.

What is claimed is:

1. An in vitro method for predicting ocular irritancy of a test substance, the method comprising:
applying an aqueous layer to a human cadaver or excised animal eye so as to form an artificial tear layer and then applying a test substance to form an overlay, incubating for a defined period of time, and then measuring the viability of the eye.

2. The method of claim 1, wherein viability is determined by one of the following assays: MTT, EZMTT, Neutral red uptake, TUNEL staining, ATP, ATP Luciferase, Luciferase, LDH, Sulforhodamine B, Resazurin, Protease (GF-AFC), RNA, Trypan blue dye exclusion, clonogenic cell survival, DNA synthesis cell proliferation, Raman microspectroscopy, live -dead biomarker stains including phalloidin, calcein-AM, propidium iodide, Hoeschst 33342, carboxyfluorescein, fluorescein diacetate, and ethidium homodimers-1 and -III or similar.

3. The method of claim 1, wherein the incubation time is identified as a period of time (e.g., 2-12 minutes) where either an ocular nonirritant (e.g., hexyl bromide CASRN 111-25-1, isoOctyl acrylate CASRN 29590-42-9, Glycerol CASRN 56-81-5, Di-iso-butyl ketone CASRN 108-83-8, 1-Bromo-4-chlorobutane CASRN 6940-78-9, 1,6-Dibromohexane CASRN 629-03-8, n-Octyl bromide CASRN 111-83-1, Propylene glycol CASRN 57-55-6, Potassium tetrafluoroborate CASRN 14075-53-7, 4,4-Methylene bis-(2,6-ditert-butyl)phenol CASRN 118-82-1, Hexane CASRN 110-54-3, 2-Ethylhexylthioglycolate CASRN 7659-86-1, isoPropyl bromide CASRN 75-26-3, 1,2,6-Hexanetriol CASRN 106-69-4, 3-Methoxy-1,2-propanediol CASRN 623-39-2, Triethylene glycol CASRN 112-27-6, Triphenyl phosphite CASRN 101-02-0, 2-Ethoxyethyl methacrylate CASRN 2370-63-0, Hexamethyldisiloxane CASRN 107-46-0, Hexyl cinnamic aldehyde CASRN 101-86-0, iso-Octylthioglycolate CASRN 25103-09-7, p-Methyl thiobenzaldehyde CASRN 3446-89-7, Triclocarban CASRN 101-20-2, Dioctyl ether CASRN 629-82-3, Di-n-propyl disulphide CASRN 629-19-6, sec-Butylbenzene CASRN 135-98-8, Isopropyl myristae CASRN 110-27-0, Polyoxyethylene hydrogenated castoroil (60E.O.) CASRN 61788-85-0, 1,3-Di-iso-propylbenzene CASRN 99-62-7, 1,9-Decadiene CASRN 1647-16-1, 2,4-Pentanediol CASRN 625-69-4, Ethylene glycol diethyl ether CASRN 629-14-1 or similar) is a FP for irritation or toxicity (as measured by a reduction in viability or other measure of toxicity) when put in direct contact with the eye, but is not a FP for irritation or toxicity (as measured by viability or other measure of toxicity) when overlayed on a aqueous layer or an ocular irritant or toxin, (such as methyl cyanoacteate CASRN 105-34-0, Isopropanol CASRN 67-63-0, Ethanol CASRN 64-17-5, Methyl acetate CASRN 79-20-9, 2-Pseudoionone CASRN 141-10-6, 1-Propoxy-2-propanol CASRN 1569-01-3, 2,6-Dichlorobenzoyl chloride CASRN 4659-45-4, Isopropylalcohol CASRN 67-30-0, Monoethanolamine CASRN 141-43-5, 4-(1,1,3,3-Tetramethylbutyl)phenol CASRN 140-66-9, Tetraethylene glycol diacrylate CASRN 17831-71-9, Hydroxyethyl acrylate CASRN 818-61-1, Tributyltin oxide CASRN 56-35-9, Parafluoraniline CASRN 371-40-4, Ethyl-2-methyl acetoacetate CASRN 609-14-3, 2-Methyl-1-pentanol CASRN 105-30-6, Sodium Hydroxide (1%) CASRN 1310-73-2, 3-Chloropropionitrile CASRN 542-76-7, n-Butanal CASRN 123-72-8, Isopropyl acetoacetate CASRN 542-08-5, Isobutyraldehyde CASRN 78-84-2, Furfuryl Alcohol CASRN 98-00-0, 2-Ethyl-1-hexanol CASRN 104-76-7, Allyl alcohol CASRN 107-18-6, Methyl ethyl ketone CASRN 78-93-3, Chlorhexidine digluconate sol. CASRN 18472-51-0, 2-Benzyloxyethanol CASRN 622-08-2, Cyclopentanol CASRN 96-41-3, n-Octanol CASRN 111-87-5, iso-butanol CASRN 78-83-1, n-Hexanol CASRN 111-27-3, Acetone CASRN 67-64-1, n-butanol CASRN 71-36-3, Butyl cellosolve CASRN 111-76-2, Methyl thioglycolate CASRN 2365-48-2, Methoxyethyl acrylate CASRN 3121-61-7, Diethylaminopropionitrile CASRN 5351-04-2, 2,2-Dimethyl Butanoic Acid CASRN 595-37-9, Cyclohexanol CASRN 108-93-0, Pyridine CASRN 110-86-1, 2-Methylbutyric acid CASRN 116-53-0, Lactic Acid CASRN 50-21-5 or similar) is identified as a true positive ocular irritant or toxins (as measured by a reduction in viability) when overlayed on an aqueous layer.

4. The method of claim 3, wherein the addition of an enzyme inhibitors: esterase inhibitors: C1 esterase inhibitor, ebelactone A, Valilactone, sulfonyl fluorides, sodium fluoride, Galantamine, Huperzine A, Tacrine or Cognex, pulegone, limonene, limonene oxide, α -terpinene, γ-terpinene, terpinen-4-ol, carvacrol, 1- and d-carvone, 1,8-cineole, p-cymene, fenchone, and pulegone-1,2-epoxide; Cytochrome P450 inhibitors: Sodium valproate, Isoniazid, Cimetidine, Ketoconazole, Fluconazole, Alcohol & Grapefruit juice, Chloramphenicol, Erythromycin, Sulfonamides, Ciprofloxacin, Omeprazole, and Metronidazole; Cytochrome P450 Inducers; Carbemazepines, Rifampicin, Alcohol, Phenytoin, Griseofulvin, Phenobarbitone, Sulphonylureas; Enzymatic inhibitors: Lepirudin, Bivalirudin, Alpha-1-proteinase inhibitor, Cyclosporine, Pravastatin, Fluvoxamine, Ramipril, Masoprocol, Carbidopa, Sildenafil, Pyrimethamine, Ticlopidine, Pantoprazole, Angiotensin Converting Enzyme Inhibitor, and Lovastatin or similar added to the aqueous layer or substance to be tested results in an increase in viability, moving the result closer to a FN and thereby demonstrating that the mechanism of viability reduction is related to enzymatic or metabolic activity.

5. An in vitro method for predicting ocular irritancy of a test substance, the method comprising:
applying the test substance to an in vitro irritancy test system in the presence of a reagent with enzymatic activity that models the metabolism of the eye, wherein the enzymatic reagent is:
(1) mixed with the test substance prior to applying to the test system,
(2) added to the test system prior to applying the test substance,
(3) or both (1) and (2);
measuring the test system response; and
predicting the ocular irritancy of the test substance based on the test system response.

6. The method in claim 5, wherein the enzymatic reagent comprises one or more compounds selected from: Crude lysate (ground up excised cornea, liver), Cytochrome P450, Esterase D, α-Naphthyl butyrate, Glutathione-S-transferases, Carboxylesterase, Carboxyl/cholinesterase, alpha/beta hydrolase, S9 fraction, Acetylcholinesterase, Neuropathy target esterase, Paraoxonases, thioesterases, lipases, cholinesterases, methylesterases, acetylhydrolase, sialate-O-acetylesterase, Acetylxylan, esterase from porcine liver and rabbit liver, esterase from Bacillus stearothermophilus, Bacillus subtilis, and Rhizopus oryzae, or similar.

7. The method of claim 5, wherein the enzymatic activity results from the activity of an intact eye and a negative control (e.g., hexyl bromide) remains negative for toxicity or irritation, but the material acted upon by the enzymatic activity of the eye changes from nontoxic to toxic or from toxic to nontoxic as a result of this activity, and the time until toxicity is measured and used to predict if the material is an ocular irritant or toxin.

8. The methods of claims 1 and 5, wherein the test material is an eye area product, drug, or agrochemical.

9. The method of claim 5, wherein the test system comprises reconstituted human corneal epithelium (RhCE).

10. The method of claim 1, wherein the test system is a DoI test system comprising excised eyes.

11. A method for reducing FP and/or FN rates of nonanimal eye irritation tests, the method comprising:
overlaying an enzymatic formulation onto a surface of a tissue, comprising reconstituted human corneal epithelium;
adding a test substance to the antioxidant formulation on the surface of differentiated eye tissue;
exposing the differentiated eye tissue to the test substance for a first period of time;
washing the surface of the differentiated eye tissue with a buffered salt solution to remove the test substance;
after a second period of time, measuring cell viability of the differentiated eye tissue; and
relating the measured cell viability to an index of irritation, which can be categorized according to established ocular irritancy classes,
wherein the FP and/or FN rate is reduced and compared to performing the method without overlaying with the enzymatic formulation.

12. The method of claims 1 and 5, wherein the established ocular irritancy classes are selected from a nonirritant, a minimal irritant, a mild irritant, and a severe irritant.

13. The method of claims 1 and 5 , wherein the established ocular irritancy classes include GHS categories NC, 2, 2B, 2A, and 1, or EPA categories IV, III, II, and I.

14. The method of claim 5, wherein the metabolic activation of the ocular toxin is confirmed by the addition of an enzyme inhibitor, comparing one or more of the following:
Esterase Inhibitors: Ebalactone, etc
General: PMS.

15. The method of claims 1 and 5, wherein the tear solution and washing solution further comprises a subsequent wash with additional salt and antioxidant formulation (e.g., an antioxidant formulation, comprising L-ascorbic acid at a concentration between 0.27 mM and 60 mM, serum albumin at a concentration of 0.05% to 10%, and dextran at a concentration of 3% to 30% in a buffered saline solution), wherein the formulation has a pH between 7-7.5.

16. The method of claims 1 and 5, wherein the eye is first overlayed with a solution of 1% bovine serum albumin and 9 g/L sodium chloride.
